(19) United States
(12) Patent Application Publication
(10) Pub. No.: US 2024/0400479 A1
(43) Pub. Date: Dec. 5, 2024

(54) METHOD FOR PRODUCING AND PURIFYING TRIFLUOROETHYLENE, AND COMPOSITION OBTAINED THEREFROM

(71) Applicant: Arkema France, Colombes (FR)

(72) Inventors: Alexandre CAMBRODON, PIERRE-BENITE CEDEX (FR); Thierry LANNUZEL, PIERRE-BENITE CEDEX (FR); Cédric LAVY, PIERRE-BENITE CEDEX (FR); Kevin HISLER, PIERRE-BENITE CEDEX (FR)

(21) Appl. No.: 18/689,101

(22) PCT Filed: Sep. 22, 2022
(86) PCT No.: PCT/FR2022/051780

§ 371 Date: Mar. 5, 2024

(30) Foreign Application Priority Data
Sep. 23, 2021 (FR) FR2110009

(51) Int. Cl. C07C 17/23; C07C 17/383

(52) U.S. Cl. CPC C07C 17/23; C07C 17/383; C07C 2523/56

(57) ABSTRACT

A method for producing trifluoroethylene in a reactor with a fixed catalytic bed comprising a catalyst, comprises the steps of: a) reacting chlorotrifluoroethylene with hydrogen in the presence of the catalyst and in the gas phase, to produce a flow of product comprising trifluoroethylene; b) treating the flow of product obtained in step a) in order to recover a stream A comprising trifluoroethylene, chlorotrifluoroethylene and an additional compound selected from the group consisting of 1,1-difluoroethylene, 1,1,1,2-tetrafluoroethane, 1,1,1-trifluoroethane and ethane; wherein the total content by weight of the additional compound is less than 0.5%; c) distilling stream A to recover a stream B comprising at least 99% by weight of trifluoroethylene and less than 0.2% by weight of one or more additional compounds. A composition comprising at least 99% by weight of trifluoroethylene and from 0.1 to 1000 ppm of ethane, and/or from 0.1 to 1000 ppm of 1,1,1,2-tetrafluoroethane, based on the total weight of the composition is disclosed.

### TECHNICAL FIELD

The present invention relates to a process for the production of hydrofluoroolefins. In particular, the present invention relates to a process for the production of trifluoroethylene (HFO-1123 or VF₃) by hydrogenolysis of chlorotrifluoroethylene. The present invention also relates to a composition comprising trifluoroethylene.

### TECHNOLOGICAL BACKGROUND OF THE INVENTION

Fluorinated olefins, such as VF_{3,} are known and are used as monomers or comonomers for the manufacture of fluorocarbon polymers exhibiting noteworthy characteristics, in particular excellent chemical resistance and good thermal resistance.

Trifluoroethylene is a gas under standard conditions of pressure and temperature. The main risks associated with the use of this product relate to its flammability, its propensity for self-polymerization when it is not stabilized, its explosiveness due to its chemical instability and its supposed sensitivity to peroxidation, by analogy with other halogenated olefins. Trifluoroethylene exhibits the distinguishing feature of being extremely flammable, with a lower explosive limit (LEL) of approximately 10% and an upper explosive limit (UEL) of approximately 30%. The major danger is, however, associated with the propensity of VF₃ to decompose violently and explosively under certain pressure conditions in the presence of an energy source, even in the absence of oxygen.

Given the main risks above, the synthesis and also the storage of VF₃ pose particular problems and impose strict safety rules throughout these processes. A known route for the preparation of trifluoroethylene uses, as starting materials, chlorotrifluoroethylene (CTFE) and hydrogen in the presence of a catalyst and in the gas phase.

A process for the production of trifluoroethylene by hydrogenolysis of CTFE in the gas phase and in the presence of a catalyst based on a metal from group VIll at atmospheric pressure and at relatively low temperatures is known from WO2013/128102.

A process for the production of trifluoroethylene is known from EP 2 993 213. Trifluoroethylene can be obtained by hydrogenolysis of chlorotrifluoroethylene or by thermal decomposition of chlorodifluoromethane and chlorofluoromethane. The production process involves carrying out a distillation stage at a pressure of 10 barg and by which the trifluoroethylene is recovered by sidestream withdrawal. Carrying out a high-pressure distillation requires that particular operating conditions to be put in place given the explosive nature of trifluoroethylene above 3 bara.

There thus exists a need to provide a simpler and safer process for the production of trifluoroethylene while maintaining high yields and selectivities.

### SUMMARY OF THE INVENTION

According to a first aspect, the present invention provides a process for the production of trifluoroethylene in a reactor equipped with a fixed catalytic bed comprising a catalyst, said process comprising the stages of:
a) reaction of chlorotrifluoroethylene with hydrogen in the presence of the catalyst and in the gas phase in order to produce a product flow comprising trifluoroethylene;
b) treatment of the product flow obtained in stage a) in order to recover a stream A comprising trifluoroethylene (HFO-1123), chlorotrifluoroethylene (HCFO-1113) and at least one additional compound selected from the group consisting of 1,1-difluoroethylene (HFO-1132a), 1,1,1,2-tetrafluoroethane (HFC-134a), 1,1,1-trifluoroethane (HFC-143a) and ethane; the total content by weight of said at least one additional compound in said stream A being less than 0.5%;
c) distillation of said stream A in order to recover a stream B comprising at least 95% by weight of trifluoroethylene and less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of the total weight of said stream B.

The present invention makes it possible to carry out a safe and efficient process for the production and purification of trifluoroethylene. The content of certain additional compounds is greatly limited while carrying out a process which is simplified compared with the prior art.

The present process also makes it possible to obtain a good yield of high-purity trifluoroethylene.

According to a preferred embodiment, stage c) of distillation of said stream A is carried out at a pressure of less than 3 bara.

According to a preferred embodiment, stage c) of distillation of said stream A is carried out in a distillation column comprising a structured packing.

According to a preferred embodiment, said stream A comprises 1,1-difluoroethylene (HFO-1132a) and 1,1,1-trifluoroethane (HFC-143a), each in a content by weight of less than 0.1%, on the basis of the total weight of said stream A.

According to a preferred embodiment, said stream A comprises 1,1,1,2-tetrafluoroethane (HFC-134a) in a content by weight of less than 0.01%, on the basis of the total weight of said stream A.

According to a preferred embodiment, said stream A comprises ethane in a content by weight of less than 0.05%, on the basis of the total weight of said stream A.

According to a preferred embodiment, said catalyst is a catalyst based on a metal from columns 8 to 10 of the Periodic Table of the Elements, preferably deposited on a support, in particular an aluminum-based support; more particularly, the catalyst comprises palladium supported on α-alumina.

According to a preferred embodiment, the chlorotrifluoroethylene and the hydrogen are in the anhydrous form.

According to second aspect, the present invention provides a composition comprising at least 99% by weight of trifluoroethylene and
from 0.1 to 1000 ppm of ethane, or
from 0.1 to 1000 ppm of 1,1,1,2-tetrafluoroethane (HFC-134a); or
from 0.1 to 1000 ppm of ethane and from 0.1 to 1000 ppm of 1,1,1,2-tetrafluoroethane (HFC-134a), on the basis of the total weight of the composition.

According to a preferred embodiment, the composition also comprises from 0.1 to 1000 ppm of 1,1,1-trifluoroethane (HFC-143a), on the basis of the total weight of the composition.

According to a preferred embodiment, the composition also comprises from 0.1 to 2000 ppm of 1,1-difluoroethylene (HFO-1132a), on the basis of the total weight of the composition.

The present invention provides a trifluoroethylene composition in which the content in additional compounds is limited. This provides many advantages in the fields of application of trifluoroethylene.

### DETAILED DESCRIPTION OF THE INVENTION

The present invention relates to a process for the production of trifluoroethylene comprising a stage of hydrogenolysis reaction of chlorotrifluoroethylene (CTFE) with hydrogen in the gas phase and preferably in the presence of a catalyst.

According to a preferred embodiment, the process according to the invention described in the present patent application is carried out continuously.

According to a preferred embodiment, in the process described in the present patent application, the hydrogen is in the anhydrous form.

According to a preferred embodiment, in the process described in the present patent application, the chlorotrifluoroethylene is in the anhydrous form.

Carrying out the processes according to the invention in the presence of anhydrous hydrogen and/or chlorotrifluoroethylene makes it possible to effectively increase the lifetime of the catalyst and thus the overall productivity of the process. The term “anhydrous” refers to a content by weight of water of less than 1000 ppm, advantageously 500 ppm, preferably of less than 200 ppm, in particular of less than 100 ppm, on the basis of the total weight of the compound under consideration.

### Catalyst

Preferably, the catalyst is based on a metal from columns 8 to 10 of the Periodic Table of the Elements. In particular, the catalyst is based on a metal selected from the group consisting of Pd, Pt, Rh, and Ru; preferably palladium.

Preferably, the catalyst is supported. The support is preferably selected from the group consisting of activated carbon, an aluminum-based support, calcium carbonate and graphite.

Preferably, the support is based on aluminum. In particular, the support is alumina. The alumina can be α-alumina. Preferably, the alumina comprises at least 90% of α-alumina. It was observed that the conversion of the hydrogenolysis reaction was improved when the alumina is an α-alumina. Thus, the catalyst is more particularly palladium supported on alumina, advantageously palladium supported on an alumina comprising at least 90% of α-alumina, preferably palladium supported on an α-alumina.

Preferably, the palladium represents from 0.01% to 5% by weight, on the basis of the total weight of the catalyst, preferably from 0.1% to 2% by weight, on the basis of the total weight of the catalyst.

In particular, said catalyst comprises from 0.01% to 5% by weight of palladium supported on alumina; preferably, the alumina comprises at least 90% of α-alumina; more preferentially, the alumina is an α-alumina.

### Activation of the Catalyst

Said catalyst is preferably activated before its use in stage a). Preferably, the activation of the catalyst is carried out at high temperature and in the presence of a reducing agent. According to a particular embodiment, the reducing agent is chosen from the group consisting of hydrogen, carbon monoxide, nitrogen monoxide, formaldehyde, C₁-C₆ alkanes and C₁-C₁₀ hydrohalocarbons, or a mixture of these; preferably hydrogen or a C₁-C₁₀ hydrohalocarbon, or a mixture of these; in particular hydrogen, chlorotrifluoroethylene, trifluoroethylene, chlorotrifluoroethane, trifluoroethane or difluoroethane, or a mixture of these. Preferably, the activation of the catalyst is carried out at a temperature of between 100° C. and 400° C., in particular at a temperature of between 150° C. and 350° C. In particular, the activation of the catalyst is carried out at a temperature of between 100° C. and 400° C., in particular at a temperature of between 150° C. and 350° C., in the presence of hydrogen as reducing agent.

### Regeneration of the Catalyst

The catalyst used in the present process can be regenerated. This regeneration stage can be carried out in a temperature range of the catalytic bed of between 90° C. and 450° C. Preferably, the regeneration stage is carried out in the presence of hydrogen. Carrying out the regeneration stage makes it possible to improve the yield of the reaction compared with the initial yield before regeneration.

According to a preferred embodiment, the regeneration stage can be carried out at a temperature of the catalytic bed of 90° C. to 300° C., preferably at a temperature of the catalytic bed of 90° C. to 250° C., more preferentially of 90° C. to 200° C., in particular of 90° C. to 175° C., more particularly at a temperature of the catalytic bed of 90° C. to 150° C. In particular, carrying out the regeneration stage at a low temperature, for example of 90° C. to 200° C. or of 90° C. to 175° C. or of 90° C. to 150° C., makes possible the desorption of compounds which are harmful to the activity of the catalyst and/or makes it possible to limit phase transitions which modify the structure of the catalyst.

According to another preferred embodiment, the regeneration stage can be carried out at a temperature of the catalytic bed of greater than 200° C., advantageously of greater than 230° C., preferably of greater than 250° C., in particular of greater than 300° C. The regeneration stage can be carried out periodically as a function of the productivity or of the conversion obtained in stage a). The regeneration stage can be advantageously carried out at a temperature of the catalytic bed of between 200° C. and 300° C., preferably between 205° C. and 295° C., more preferentially between 210° C. and 290° C., in particular between 215° C. and 290° C., more particularly between 220° C. and 285° C., in a favored way between 225° C. and 280° C., in a more favored way between 230° C. and 280° C. Alternatively, the regeneration stage can be carried out at a temperature of between 300° C. and 450° C., preferably between 300° C. and 400° C. The regenerated catalyst can be reused in stage a) of the present process.

### Hydrogenolysis Reaction

The present invention comprises, as mentioned above, a stage of hydrogenolysis reaction of chlorotrifluoroethylene (CTFE) with hydrogen in order to produce a stream comprising trifluoroethylene. The hydrogenolysis stage is carried out in the presence of a catalyst and in the gas phase. Preferably, the hydrogenolysis stage is carried out in the presence of a preactivated catalyst and in the gas phase. The hydrogenolysis stage consists in simultaneously introducing hydrogen, CTFE and optionally an inert gas, such as nitrogen, in the gas phase and in the presence of said catalyst, which is preferably activated.

Preferably, said stage a) is carried out at a temperature of the fixed catalytic bed of between 50° C. and 250° C. Said stage a) can be carried out at a temperature of the fixed catalytic bed of between 50° C. and 240° C., advantageously between 50° C. and 230° C., preferably between 50° C. and 220° C., more preferentially between 50° C. and 210° C., in particular between 50° C. and 200° C. Said stage a) can also be carried out at a temperature of the fixed catalytic bed of between 60° C. and 250° C., advantageously between 70° C. and 250° C., preferably between 80° C. and 250° C., more preferentially between 90° C. and 250° C., in particular between 100° C. and 250° C., more particularly between 120° C. and 250° C. Said stage a) can also be carried out at a temperature of the fixed catalytic bed of between 60° C. and 240° C., advantageously between 70° C. and 230° C., preferably between 80° C. and 220° C., more preferentially between 90° C. and 210° C., in particular between 100° C. and 200° C., more particularly between 100° C. and 180° C., favorably between 100° C. and 160° C., particularly preferably between 120° C. and 160° C.

The H₂/CTFE molar ratio is of between 0.5/1 and 2/1, and preferably of between 1/1 and 1.2/1. If an inert gas, such as nitrogen, is present in stage a), the nitrogen/H₂ molar ratio is of between 0/1 and 2/1 and preferably of between 0/1 and 1/1.

Stage a) is preferably carried out at a pressure of 0.05 MPa to 1.1 MPa, more preferentially of 0.05 MPa to 0.5 MPa, in particular at atmospheric pressure.

The contact time, calculated as being the ratio of the volume, in liters, of catalyst to the total flow rate of the gas mixture, in standard liters per second, at the inlet of the reactor, is of between 1 and 60 seconds, preferably between 5 and 45 seconds, in particular between 10 and 30 seconds, more particularly between 15 and 25 seconds.

The hydrogenolysis stage (stage a)) of the present process results in the production of a product flow comprising trifluoroethylene. Said product flow can also comprise unreacted hydrogen and unreacted chlorotrifluoroethylene. Said product flow can also contain 1,1-difluoroethylene, 1,1,1,2-tetrafluoroethane, 1,1,1-trifluoroethane or ethane. Said product flow can also comprise HCl or HF or a mixture of the two. Carrying out this stage a) makes it possible to produce trifluoroethylene containing a reduced content of 1,1-difluoroethylene, 1,1,1,2-tetrafluoroethane, 1,1,1-trifluoroethane and/or ethane. This facilitates the stages of treatment of the reaction flow and results in a better overall yield of the process.

### Treatment of the Reaction Flow

According to stage b) of the present process, the product flow resulting from stage a) is treated in order to recover a stream A comprising trifluoroethylene (HFO-1123), chlorotrifluoroethylene (HCFO-1113) and at least one additional compound selected from the group consisting of 1,1-difluoroethylene (HFO-1132a), 1,1,1,2-tetrafluoroethane (HFC-134a), 1,1,1-trifluoroethane (HFC-143a) and ethane. Said stream A can thus comprise one, two, three or the four additional compounds mentioned above.

According to a preferred embodiment, the total content by weight of said at least one additional compound in said stream A is less than 0.5%, advantageously less than 0.4%, preferably less than 0.3%, more preferentially less than 0.2%, on the basis of the total weight of said stream A. Thus, the combined additional compounds present in said stream A represent a content by weight of less than 0.5%, advantageously of less than 0.4%, preferably of less than 0.3%, more preferentially of less than 0.2%, on the basis of the total weight of said stream A.

According to a preferred embodiment, said stream A comprises 1,1,1,2-tetrafluoroethane (HFC-134a) in a content by weight of less than 0.05%, advantageously of less than 0.025%, preferably of less than 0.01%, on the basis of the total weight of said stream A.

According to a preferred embodiment, said stream A comprises ethane in a content by weight of less than 0.1%, preferably of less than 0.05%, in particular of less than 0.025%, on the basis of the total weight of said stream A.

According to a preferred embodiment, said stream A comprises 1,1-difluoroethylene (HFO-1132a) in a content by weight of less than 0.2%, advantageously of less than 0.15%, preferably of less than 0.1%, in particular of less than 0.08%, more particularly of less than 0.075%, on the basis of the total weight of said stream A.

According to a preferred embodiment, said stream A comprises 1,1,1-trifluoroethane in a content by weight of less than 0.2%, advantageously of less than 0.15%, preferably of less than 0.1%, on the basis of the total weight of said stream A.

According to a preferred embodiment, said stream A comprises 1,1-difluoroethylene (HFO-1132a) and 1,1,1-trifluoroethane (HFC-143a), each in a content by weight of less than 0.2%, preferably of less than 0.1%, on the basis of the total weight of said stream A.

According to a preferred embodiment, said stream A comprises 1,1-difluoroethylene (HFO-1132a) and 1,1,1-trifluoroethane (HFC-143a), each in a content by weight of less than 0.2%, preferably of less than 0.1%, on the basis of the total weight of said stream A; and said stream A comprises ethane in a content by weight of less than 0.1%, preferably of less than 0.05%, on the basis of the total weight of said stream A.

According to a preferred embodiment, said stream A comprises 1,1-difluoroethylene (HFO-1132a) and 1,1,1-trifluoroethane (HFC-143a), each in a content by weight of less than 0.2%, preferably of less than 0.1%, on the basis of the total weight of said stream A; and said stream A comprises 1,1,1,2-tetrafluoroethane (HFC-134a) in a content by weight of less than 0.05%, advantageously of less than 0.025%, preferably of less than 0.01%, on the basis of the total weight of said stream A.

According to a preferred embodiment, said stream A comprises 1,1-difluoroethylene (HFO-1132a) and 1,1,1-trifluoroethane (HFC-143a), each in a content by weight of less than 0.2%, preferably of less than 0.1%, on the basis of the total weight of said stream A; and said stream A comprises 1,1,1,2-tetrafluoroethane (HFC-134a) in a content by weight of less than 0.05%, advantageously of less than 0.025%, preferably of less than 0.01%, on the basis of the total weight of said stream A; and said stream A comprises ethane in a content by weight of less than 0.1%, preferably of less than 0.05%, on the basis of the total weight of said stream A.

According to a preferred embodiment, in said stream A, the content by weight of trifluoroethylene is greater than 10%, advantageously greater than 15%, preferably greater than 20%, in particular greater than 25%, more particularly greater than 30%, on the basis of the total weight of said stream A.

According to a preferred embodiment, in said stream A, the content by weight of chlorotrifluoroethylene is less than 70%, advantageously less than 65%, preferably less than 60%, in particular less than 55%, on the basis of the total weight of said stream A. Preferably, in said stream A, the content by weight of chlorotrifluoroethylene is greater than 1%, preferably greater than 5%, on the basis of the total weight of said stream A. Thus, said stream A can comprise trifluoroethylene (HFO-1123), chlorotrifluoroethylene (HCFO-1113), 1,1-difluoroethylene (HFO-1132a), 1,1,1,2-tetrafluoroethane (HFC-134a), 1,1,1-trifluoroethane (HFC-143a) and ethane in any one of the contents by weight expressed above.

Thus, according to a particular embodiment, said stream A comprises:
trifluoroethylene in a content by weight of greater than 10%, advantageously of greater than 15%, preferably of greater than 20%, in particular of greater than 25%, more particularly of greater than 30%, on the basis of the total weight of said stream A;
chlorotrifluoroethylene in a content by weight of less than 70%, advantageously of less than 65%, preferably of less than 60%, in particular of less than 55%, on the basis of the total weight of said stream A, and optionally of greater than 1%, preferably of greater than 5%, on the basis of the total weight of said stream A;
1,1,1,2-tetrafluoroethane (HFC-134a) in a content by weight of less than 0.05%, advantageously of less than 0.025%, preferably of less than 0.01%, on the basis of the total weight of said stream A;
1 ethane in a content by weight of less than 0.1%, preferably of less than 0.05%, on the basis of the total weight of said stream A;
1,1-difluoroethylene (HFO-1132a) in a content by weight of less than 0.2%, advantageously of less than 0.15%, preferably of less than 0.1%, in particular of less than 0.08%, on the basis of the total weight of said stream A; and
1,1,1-trifluoroethane in a content by weight of less than 0.2%, advantageously of less than 0.15%, preferably of less than 0.1%, on the basis of the total weight of said stream A;
   and the total content by weight of 1,1,1,2-tetrafluoroethane, ethane, 1,1-difluoroethylene and 1,1,1-trifluoroethane in said stream A is less than 0.5%, advantageously less than 0.4%, preferably less than 0.3%, more preferentially less than 0.2%, on the basis of the total weight of said stream A.

When one of said additional compounds selected from the group consisting of 1,1-difluoroethylene (HFO-1132a), 1,1,1,2-tetrafluoroethane (HFC-134a), 1,1,1-trifluoroethane (HFC-143a) and ethane is present in said stream A, this (or these) is (are) present in a content by weight of greater than 0.01 ppm, preferably of greater than 0.1 ppm, on the basis of the total weight of said stream A.

According to a particular embodiment, the treatment stage b) of the present process can comprise the stages of:
i) removal of HF and/or HCl from said product flow obtained in stage a) in order to form a gas mixture;
ii) drying of the gas mixture resulting from stage i);
iii) treatment of the gas mixture dried in stage ii) in order to remove the hydrogen and optionally inert gases and to form said stream A.

Preferably, said stream A is gaseous.

The paragraph below describes stages i) to iii) in detail

The product flow resulting from stage a) is recovered at the reactor outlet in gaseous form.

Preferably, at the outlet of the hydrogenolysis reactor, the product flow is first of all treated in order to remove HCl and HF. The product flow is passed through water in a washing column, then washed with a dilute base, such as NaOH or KOH. The remainder of the gas mixture, consisting of the unconverted reactants (H₂ and CTFE), dilution nitrogen (if present), trifluoroethylene and additional compounds mentioned above, is directed to a dryer in order to remove the traces of washing water. Drying can be carried out using products such as calcium, sodium or magnesium sulfate, calcium chloride, potassium carbonate, silica gel or zeolites. In one embodiment, a molecular sieve (zeolite), such as siliporite, is used for the drying. The gas mixture, thus dried, is subjected to a stage of separation of the hydrogen and inert substances from the remainder of the other products present in the gas mixture by absorption/desorption in the presence of an alcohol comprising from 1 to 4 carbon atoms and preferably ethanol, at atmospheric pressure and at a temperature below ambient temperature, preferably of less than 10° C. and more preferably still at a temperature of −25° C., for the absorption. In one embodiment, the absorption of the organic substances is carried out in a countercurrent column with ethanol cooled to −25° C. The ethanol flow rate is adjusted according to the flow rate of organic substances to be absorbed. The hydrogen and inert gases, which are insoluble in ethanol at this temperature, are removed at the absorption column top. The organic substances are subsequently recovered in the form of said stream A, by heating the ethanol to its boiling point (desorption), in order to be subsequently distilled.

Stages a) and b) of the present process thus make it possible to limit the content of the additional compound(s) in the stream A, which makes it easier to carry out stage c) described below (in particular by carrying out this stage at low pressure).

According to stage c), said stream A thus obtained is distilled in order to form and recover a stream B comprising trifluoroethylene and one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane. Said stream B can comprise one, two, three or the four additional compounds mentioned above.

According to a preferred embodiment, stage c) of distillation of said stream A is carried out at a pressure of less than 3 bara, preferably at a pressure of between 0.5 and 3 bara, in particular at a pressure of between 0.9 and 2 bara. Carrying out a distillation at a pressure of less than 3 bara makes it possible to render the process more secure given the explosive nature of trifluoroethylene above 3 bara.

Preferably, stage c) of distillation of said stream A is carried out in a distillation column comprising a structured packing. It has been observed that a structured packing makes it possible to obtain a more efficient distillation stage c). The structured packing can be made of a metallic material.

Said stream B is preferably recovered at the top of the distillation column. Before being recovered, the stream B can optionally be partially condensed at the top of the distillation column. When the partial condensation is carried out, the stream B is brought to a temperature of −50° C. to −70° C. The temperature is adjusted according to the pressure applied in stage c).

The partial condensation makes it possible to improve the efficiency of the distillation by limiting the content of additional compounds in the stream B.

The distillation of said stream A also results in the formation of a stream C comprising chlorotrifluoroethylene, preferably recovered at the bottom of the distillation column. Said stream C can be recycled in stage a) after an optional purification treatment.

Said stream B can comprise at least 95% of trifluoroethylene, advantageously at least 96%, preferably at least 97%, in particular at least 98%, more particularly at least 99%, by weight, on the basis of the total weight of said stream B.

Preferably, said stream B also comprises less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of the total weight of said stream B.

Thus, said stream B can comprise at least 95% by weight of trifluoroethylene and less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of the total weight of said stream B.

Advantageously, said stream B can comprise at least 96% by weight of trifluoroethylene and less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of the total weight of said stream B.

Preferably, said stream B can comprise at least 97% by weight of trifluoroethylene and less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of the total weight of said stream B.

More preferentially, said stream B can comprise at least 98% by weight of trifluoroethylene and less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of the total weight of said stream B.

In particular, said stream B can comprise at least 99% by weight of trifluoroethylene and less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of the total weight of said stream B.

Preferably, said stream B comprises ethane in a content by weight of less than 500 ppm, more preferentially of less than 200 ppm, in particular of less than 100 ppm, more particularly of less than 50 ppm, in a favored way of less than 10 ppm, on the basis of the total weight of said stream B.

Preferably, said stream B comprises 1,1,1,2-tetrafluoroethane in a content by weight of less than 500 ppm, more preferentially of less than 200 ppm, in particular of less than 100 ppm, more particularly of less than 50 ppm, in a favored way of less than 10 ppm, on the basis of the total weight of said stream B. Said stream B can also be devoid of 1,1,1,2-tetrafluoroethane.

Preferably, said stream B comprises 1,1,1-trifluoroethane in a content by weight of less than 1000 ppm, more preferentially of less than 750 ppm, in particular of less than 500 ppm, more particularly of less than 250 ppm, on the basis of the total weight of said stream B.

Preferably, said stream B comprises 1,1-difluoroethylene in a content by weight of less than 2000 ppm, more preferentially of less than 1500 ppm, in particular of less than 1000 ppm, on the basis of the total weight of said stream B.

When one of said additional compounds selected from the group consisting of 1,1-difluoroethylene (HFO-1132a), 1,1,1,2-tetrafluoroethane (HFC-134a), 1,1,1-trifluoroethane (HFC-143a) and ethane is present in said stream B, this (or these) is (are) present in a content by weight of greater than 0.01 ppm, preferably of greater than 0.1 ppm, on the basis of the total weight of said stream B.

### Composition

According to a second aspect, the present invention provides trifluoroethylene compositions of high purity.

Said composition comprises at least 99% by weight of trifluoroethylene and from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of ethane, on the basis of the total weight of the composition.

Said composition can also comprise at least 99% by weight of trifluoroethylene and from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of 1,1,1,2-tetrafluoroethane (HFC-134a), on the basis of the total weight of the composition.

Said composition can also comprise at least 99% by weight of trifluoroethylene and:
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of ethane; and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of 1,1,1,2-tetrafluoroethane (HFC-134a), on the basis of the total weight of the composition.

According to a preferred embodiment, any one of the above compositions also comprises from 0.1 to 1000 ppm, preferably from 0.1 to 750 ppm, in particular from 0.1 to 500 ppm, more preferentially from 0.1 to 250 ppm, of 1,1,1-trifluoroethane (HFC-143a), on the basis of the total weight of the composition.

According to a preferred embodiment, any one of the above compositions also comprises from 0.1 to 2000 ppm, preferably from 0.1 to 1500 ppm, in particular from 0.1 to 1000 ppm, of 1,1-difluoroethylene (HFO-1132a), on the basis of the total weight of the composition.

Said composition can comprise at least 99% by weight of trifluoroethylene and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of ethane,
from 0.1 to 1000 ppm, preferably from 0.1 to 750 ppm, in particular from 0.1 to 500 ppm, more preferentially from 0.1 to 250 ppm, of 1,1,1-trifluoroethane (HFC-143a), on the basis of the total weight of the composition.

Said composition can comprise at least 99% by weight of trifluoroethylene and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of 1,1,1,2-tetrafluoroethane (HFC-134a),
from 0.1 to 1000 ppm, preferably from 0.1 to 750 ppm, in particular from 0.1 to 500 ppm, more preferentially from 0.1 to 250 ppm, of 1,1,1-trifluoroethane (HFC-143a), on the basis of the total weight of the composition.

Said composition can comprise at least 99% by weight of trifluoroethylene and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of ethane,
from 0.1 to 2000 ppm, preferably from 0.1 to 1500 ppm, in particular from 0.1 to 1000 ppm, of 1,1-difluoroethylene (HFO-1132a), on the basis of the total weight of the composition.

Said composition can comprise at least 99% by weight of trifluoroethylene and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of 1,1,1,2-tetrafluoroethane (HFC-134a),
from 0.1 to 2000 ppm, preferably from 0.1 to 1500 ppm, in particular from 0.1 to 1000 ppm, of 1,1-difluoroethylene (HFO-1132a), on the basis of the total weight of the composition.

Thus, according to a preferred embodiment, said composition comprises at least 99% by weight of trifluoroethylene and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of ethane,
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of 1,1,1,2-tetrafluoroethane (HFC-134a),
from 0.1 to 1000 ppm, preferably from 0.1 to 750 ppm, in particular from 0.1 to 500 ppm, more preferentially from 0.1 to 250 ppm, of 1,1,1-trifluoroethane (HFC-143a), on the basis of the total weight of the composition.

Thus, according to another preferred embodiment, said composition comprises at least 99% by weight of trifluoroethylene and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of ethane,
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of 1,1,1,2-tetrafluoroethane (HFC-134a),
from 0.1 to 2000 ppm, preferably from 0.1 to 1500 ppm, in particular from 0.1 to 1000 ppm, of 1,1-difluoroethylene (HFO-1132a), on the basis of the total weight of the composition.

According to another particular embodiment, said composition comprises at least 99% by weight of trifluoroethylene and
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of ethane,
from 0.1 to 1000 ppm, advantageously from 0.1 to 500 ppm, preferably from 0.1 to 200 ppm, more preferentially from 0.1 to 100 ppm, in particular from 0.1 to 50 ppm, more particularly from 0.1 to 10 ppm, of 1,1,1,2-tetrafluoroethane (HFC-134a),
from 0.1 to 1000 ppm, preferably from 0.1 to 750 ppm, in particular from 0.1 to 500 ppm, more preferentially from 0.1 to 250 ppm, of 1,1,1-trifluoroethane (HFC-143a),
from 0.1 to 2000 ppm, preferably from 0.1 to 1500 ppm, in particular from 0.1 to 1000 ppm, of 1,1-difluoroethylene (HFO-1132a), on the basis of the total weight of the composition.

Said compositions above can be obtained by the process according to the present invention.

### EXAMPLE

25 cm³ of catalyst (0.2% of palladium supported on α-alumina) were introduced into a tubular reactor consisting of a stainless steel tube with a length of 1200 mm over a diameter of 25 mm, and equipped with a jacket. The catalyst, thus charged, was subsequently activated in the following way: the reaction tube was placed in a tube furnace and was fed with a stream of hydrogen (from 0.05 to 0.1 mol per gram of catalyst). The catalytic bed was then heated to a temperature of 200° C. to 250° C. with a temperature gradient of 0.2° C./min. After this activation period, the tube was cooled to ambient temperature and then was isolated in order to be subsequently installed on a hydrogenolysis test bench. The reactor was fed with 1 mol/h of CTFE and 1 mol/h of hydrogen in the anhydrous form. It is also possible to feed the reactors with an inert gas (in this instance nitrogen). The temperature of the catalytic bed was between 100° C. and 130° C. The contact time, calculated as being the ratio of the volume in liters of catalyst to the sum of the flow rates of the reactants in standard liters per second, was of the order of 22 seconds.

The gases resulting from the reaction are introduced into a column for scrubbing out the hydracids consisting of a tube made of fluoropolymer with a length of 355 mm and with a diameter of 40 mm packed with rings made of fluoropolymer with a diameter of 4 mm and with a length of 5 mm. The scrubbing column is fed continuously with water at a flow rate of 10 l/h. The water laden with hydracid is continuously removed at the bottom of the scrubbing column. The reaction products, thus freed from the hydracids, are subsequently sent to a drying section consisting of two metal tubes made of stainless steel with a length of 800 mm and with a diameter of 50 mm, mounted in series, filled with molecular sieve of the siliporite 3A type. The gases, thus dried, are subsequently sent to an absorption column consisting of a metal tube made of stainless steel with a length of 700 mm and with a diameter of 40 mm equipped with a jacket and packed with glass rings with a diameter of 4.3 mm and with a length of 4.5 mm. The absorption column is fed at the top with ethanol via a pump, the flow rate of which is 8 liters/hour. The jacket of the absorption column is fed with a heat-exchange fluid at −25° C. The hydrogen and the inert substances exit at the top of the absorption column, whereas the products of the reaction, dissolved in the ethanol, exit at the bottom of the column and are sent to a desorption section consisting of a glass column with a length of 250 mm and with a diameter of 18 mm, packed with glass rings with a diameter of 4.3 mm and with a length of 4.5 mm, and of a 1 liter round-bottomed glass flask where the ethanol is brought to boiling point using a heating mantle. The organic products resulting from the reaction are evaporated and leave the desorption section via the column top, whereas the ethanol, freed from the organic substances, is picked up by the pump in order to be fed at the top of the absorption column.

The mixture of organic products resulting from the desorption section is subsequently sent to a distillation column comprising a Sulzer EX or Sulzer DX structured packing. The rectification section is equivalent to 12 to 13 theoretical stages and the stripping section is equivalent to 1 theoretical stage. The stream A entering the distillation column comprises between 35% and 40% of trifluoroethylene, from 45% to 55% of chlorotrifluoroethylene, from 0.01% to 0.02% of ethane, from 0.05% to 0.1% of 1,1-difluoroethylene, from 0.05% to 0.1% of 1,1,1-trifluoroethane and from 0.005% to 0.01% of 1,1,1,2-tetrafluoroethane. This distillation stage is carried out at a pressure of between 0.8 and 1.2 bara. The stream B is recovered at the top of the distillation column. The stream B comprises 99.5% of trifluoroethylene, 0.1% of 1,1-difluoroethylene (HFO-1132a), 0.02% of 1,1,1-trifluoroethane (HFC-143a) and 0.0002% of ethane and less than 1 ppm of 1,1,1,2-tetrafluoroethane (HFC-134a).

1-11. (canceled)

12. A process for production of trifluoroethylene in a reactor equipped with a fixed catalytic bed comprising a catalyst, said process comprising the stages of:
a) reaction of chlorotrifluoroethylene with hydrogen in the presence of the catalyst and in a gas phase in order to produce a product flow comprising trifluoroethylene;
b) treatment of the product flow obtained in stage a) in order to recover a stream A comprising trifluoroethylene (HFO-1123), chlorotrifluoroethylene (HCFO-1113) and at least one additional compound selected from the group consisting of 1,1-difluoroethylene (HFO-1132a), 1,1, 1,2-tetrafluoroethane (HFC-134a), 1,1,1-trifluoroethane (HFC-143a) and ethane; a total content by weight of said at least one additional compound in said stream A being less than 0.5%; and
c) distillation of said stream A in order to recover a stream B comprising at least 95% by weight of trifluoroethylene and less than 0.2% by weight of one or more additional compound(s) selected from the group consisting of 1,1-difluoroethylene (HFO-1132a), 1,1,1,2-tetrafluoroethane (HFC-134a), 1,1,1-trifluoroethane (HFC-143a) and ethane, on the basis of a total weight of said stream B.

13. The process as claimed in claim 12, characterized in that stage c) of distillation of said stream A is carried out at a pressure of less than 3 bara.

14. The process as claimed in claim 12, characterized in that stage c) of distillation of said stream A is carried out in a distillation column comprising a structured packing.

15. The process as claimed in claim 12, characterized in that said stream A comprises 1,1-difluoroethylene (HFO-1132a) and 1,1,1-trifluoroethane (HFC-143a), each in a content by weight of less than 0.1%, on the basis of the total weight of said stream A.

16. The process as claimed in claim 12, characterized in that said stream A comprises 1,1,1,2-tetrafluoroethane (HFC-134a) in a content by weight of less than 0.01%, on the basis of the total weight of said stream A.

17. The process as claimed in claim 12, characterized in that said stream A comprises ethane in a content by weight of less than 0.05%, on the basis of the total weight of said stream A.

18. The process as claimed in claim 12, characterized in that said catalyst is a catalyst based on a metal from columns 8 to 10 of the Periodic Table of the Elements.

19. The process as claimed in claim 12, characterized in that the chlorotrifluoroethylene and the hydrogen are in anhydrous form.

20. A composition comprising at least 99% by weight of trifluoroethylene and
1 from 0.1 to 1000 ppm of ethane, or
from 0.1 to 1000 ppm of 1,1,1,2-tetrafluoroethane (HFC-134a); or
from 0.1 to 1000 ppm of ethane and from 0.1 to 1000 ppm of 1,1,1,2-tetrafluoroethane (HFC-134a), on the basis of a total weight of the composition.

21. The composition as claimed in claim 20, characterized in that the composition also comprises from 0.1 to 1000 ppm of 1,1,1-trifluoroethane (HFC-143a), on the basis of the total weight of the composition.

22. The composition as claimed in claim 20, characterized in that the composition also comprises from 0.1 to 2000 ppm of 1.1-difluoroethylene (HFO-1132a), on the basis of the total weight of the composition.
